(19) United States
(12) Patent Application Publication
(10) Pub. No.: US 2009/0188784 A1
(43) Pub. Date: Jul. 30, 2009

(54) BIO-SENSORS INCLUDING NANOCHANNEL INTEGRATED 3-DIMENSIONAL METALLIC NANOWIRE GAP ELECTRODES, MANUFACTURING METHOD THEREOF, AND BIO-DISK SYSTEM COMPRISING THE BIO-SENSORS

(76) Inventors: Byung Chul Lee, Seoul (KR); Sung Wook Moon, Namyangju (KR)

Correspondence Address: PERKINS COIE LLP, P.O. BOX 1208, SEATTLE, WA, 98111-1208, US

(21) Appl. No.: 12/199,793

(22) Filed: Aug. 27, 2008

(30) Foreign Application Priority Data
Jan. 30, 2008 (KR) 2008-9649

(51) Int. Cl. C23C 14/00; G01N 33/487

(52) U.S. Cl. 204/192.1; 204/403.01

(57) ABSTRACT

There are provided a bio-sensor including nanochannel-integrated 3-dimensional metallic nanowire gap electrodes, a manufacturing method thereof, and a bio-disk system comprising the bio-sensor. The bio-sensor includes an upper substrate block having a plurality of metallic nanowires formed on a lower surface thereof and including an injection port through which a biomaterial-containing sample is injected; a lower substrate block having a plurality of metallic nanowires formed on an upper surface thereof; and a supporting unit supporting the upper and lower substrate blocks so that the upper and lower substrate blocks can be disposed spaced apart at a predetermined distance to form a nanochannel, wherein the metallic nanowires formed on the upper and lower substrate blocks are combined to form 3-dimensional metallic nanowire gap electrodes.

### CROSS-REFERENCE TO RELATED APPLICATIONS

This application claims the priority of Korean Patent Application No. 2008-9649 filed on Jan. 30, 2008, in the Korean Intellectual Property Office, the disclosure of which is incorporated herein by reference.

### BACKGROUND OF THE INVENTION

1. Field of the Invention

The present invention relates to a bio-sensor including nanochannel-integrated 3-dimensional metallic nanowire gap electrodes, a manufacturing method thereof, and a bio-disk system comprising the bio-sensors, and more particularly, to a method of manufacturing a bio-sensor including 3-dimensional metallic nanowire gap electrodes, the method including: forming metallic nanowires on upper and lower substrates, arranging the upper and lower substrates using the metallic nanowires and attaching the upper and lower substrates, a bio-sensor manufactured using the method, and a bio-disk system to detect a biomaterial using the bio-sensor.

2. Description of the Related Art

For the past ten years, a keen interest has been increasingly taken in the human lift extension and the early diagnosis of diseases, and a nano-bio technology has appeared as one of cutting-edge fusion technologies to solve the problems regarding the human lift extension and the early diagnosis of diseases. Among them, a nano-biosensor/chip has become known as the key to the nano-bio technology, and therefore many developed countries have ardently attempted to enhance the sensitivity and accuracy of the nano-biosensor/chip.

Since the sizes of biomaterials such as DNA, RNA, PNA, proteins, or the like are in the range from several nanometers to several hundred nanometers, both bio-sensors to detect the biomaterials and channels as the passages through which the biomaterials to be detected flow should be in the range of nanometers in order to enhance the sensitivity and accuracy of the bio-sensors. In particular, the use of nanogaps or nanowires in the manufacture of highly sensitive sensors makes it possible to detect the biomaterials more effectively.

FIG. 1A is a schematic view illustrating flat metallic nanowire electrodes that have been used in conventional nanobiosensors. As shown in FIG. 1A, the conventional nanobiosensors function to detect a biomaterial by measuring the antigen-antibody reaction when antibody 1 anchored onto metallic nanowires 10 arranged 2-dimensionally on a substrate block encounters a sample including antigen 2. In this case, when the probability that the antigen binds to the antibody is small and a concentration of the antigen is low, the sensitivity of the antibody to the antigen may be low due to the narrow possibility to detect a biomaterial. That is, the conventional nanobiosensors have their limits to detect the biomaterial at high sensitivity and high efficiency. Therefore, the very small probability that a sensor block to detect a biomaterial is in contact with the biomaterial requires an extremely large amount of a sample including a biomaterial in order to enhance the contact probability of the sensor block to the biomaterial.

### SUMMARY OF THE INVENTION

The present invention is designed to solve the problems of the prior art, and therefore it is an object of the present invention to provide a method of manufacturing a bio-sensor including 3-dimensional metallic nanowire gap electrodes, the method including: forming metallic nanowires on upper and lower substrates, arranging the upper and lower substrates using the metallic nanowires, and attaching the upper and lower substrates, a bio-sensor manufactured using the method, and a bio-disk system to detect a biomaterial using the bio-sensor.

According to an aspect of the present invention, there is provided a bio-sensor including 3-dimensional metallic nanowire gap electrodes, including an upper substrate block having a plurality of metallic nanowires formed on a lower surface thereof and including an injection port through which a sample including a biomaterial is injected; a lower substrate block having a plurality of metallic nanowires formed on an upper surface thereof; and a supporting unit supporting the upper and lower substrate blocks with the upper and lower substrate blocks being disposed spaced apart at a predetermined distance to form a nanochannel, wherein the metallic nanowires formed on the upper and lower substrate blocks are combined to form 3-dimensional metallic nanowire gap electrodes.

According to another aspect of the present invention, there is also provided a method of manufacturing a bio-sensor including 3-dimensional metallic nanowire gap electrodes, and this method includes: (a) forming a metal electrode on an upper surface of a lower substrate; (b) patterning a nanochannel on a resist to determine a width and a length of the nanochannel, the resist being applied onto a lower surface of an upper substrate; (c) etching the nanochannel using, as a mask, the pattern formed in operation (b); (d) forming metal electrodes on the nanochannel formed in operation (c); (e) arranging the upper and lower substrates using the metal electrodes formed on the upper and lower substrates; and (f) attaching the upper and lower substrates arranged in operation (e).

According to still another aspect of the present invention, there is also provided a method of manufacturing a bio-sensor including 3-dimensional metallic nanowire gap electrodes, the method including: (a) forming a plurality of metallic nanowires on an upper surface of a lower substrate; (b) forming a plurality of metallic nanowires on a lower surface of an upper substrate; (c) spin-coating a polymer onto the upper surface of the lower substrate to form a nanochannel; (d) determining a width and a length of the nanochannel and etching the polymer using a mask pattern; (e) arranging the upper and lower substrates using the metallic nanowires formed on the upper and lower substrates; and (f) attaching the upper and lower substrates arranged in operation (e).

According to still another aspect of the present invention, there is also provided a method of manufacturing a bio-sensor including 3-dimensional metallic nanowire gap electrodes, the method including: (a) forming a plurality of metallic nanowires on an upper surface of a lower substrate; (b) forming a plurality of metallic nanowires on a lower surface of an upper substrate; (c) spin-coating a polymer on the upper surface of the lower substrate to form a nanochannel; (d) arranging the upper and lower substrates using the metallic nanowires formed on the upper and lower substrates; (e) attaching the upper and lower substrates arranged in operation (d); and (f) determining a width and a length of the nanochannel and removing the polymer by UV exposure using the mask pattern.

According to yet another aspect of the present invention, there is also provided a bio-disk system for detecting a biomaterial from an injected sample using the bio-sensor as defined in any one of claims 1 to 9. In this case, the bio-sensor may be disposed in a thin disk-type body selected from the group consisting of CD-ROMs, DVDs, bio CDs and bio DVDs.

### BRIEF DESCRIPTION OF THE DRAWINGS

The above and other aspects, features and other advantages of the present invention will be more clearly understood from the following detailed description taken in conjunction with the accompanying drawings, in which:

FIG. 1A is a schematic view illustrating flat metallic nanowire electrodes that have been used in conventional nanobiosensors.

FIG. 1B is a schematic view illustrating 3-dimensional metallic nanowire gap electrodes used in a nanobiosensor according to one exemplary embodiment of the present invention.

FIGS. 2A and 2B are a cross-sectional view and a top plane view, respectively, illustrating a bio-sensor including 3-dimensional metallic nanowire gap electrodes according to one exemplary embodiment of the present invention.

FIGS. 3A and 3B are a cross-sectional view and a top plane view, respectively, illustrating a bio-sensor including 3-dimensional metallic nanowire gap electrodes according to another exemplary embodiment of the present invention.

FIGS. 4A and 4B are a cross-sectional view and a top plane view, respectively, illustrating a bio-sensor including 3-dimensional metallic nanowire gap electrodes according to still another exemplary embodiment of the present invention.

FIGS. 5A and 5B are a cross-sectional view and a top plane view, respectively, illustrating a bio-sensor including 3-dimensional metallic nanowire gap electrodes according to still another exemplary embodiment of the present invention.

FIGS. 6A and 6B are a cross-sectional view and a top plane view, respectively, illustrating a bio-sensor including 3-dimensional metallic nanowire gap electrodes according to yet another exemplary embodiment of the present invention.

FIG. 7 is a flow chart illustrating a method of manufacturing a bio-sensor including 3-dimensional metallic nanowire gap electrodes according to one exemplary embodiment of the present invention.

FIG. 8 is a flow chart illustrating a method of manufacturing a bio-sensor including 3-dimensional metallic nanowire gap electrodes according to another exemplary embodiment of the present invention.

FIG. 9 is a flow chart illustrating a method of manufacturing a bio-sensor including 3-dimensional metallic nanowire gap electrodes according to still another exemplary embodiment of the present invention.

FIG. 10A is a configurational view illustrating a bio-disk system including the bio-sensor according to one exemplary embodiment of the present invention.

FIG. 10B is a configurational view illustrating a bio-sensor including the metallic nanowire gap electrodes according to one exemplary embodiment of the present invention.

FIG. 10C is a plane view illustrating the bio-disk system arranged on a disk-type body.

### DETAILED DESCRIPTION OF THE PREFERRED EMBODIMENT

Exemplary embodiments of the present invention will now be described in detail with reference to the accompanying drawings. Although shown in different drawings, it should be understood that the same components in the drawings have the same reference numerals. For the exemplary embodiments of the present invention, detailed descriptions of known functions and constructions that are related to the present invention are omitted for clarity when they are unnecessarily proven to makes the gist of the present invention unnecessarily unclear.

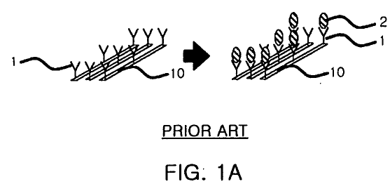

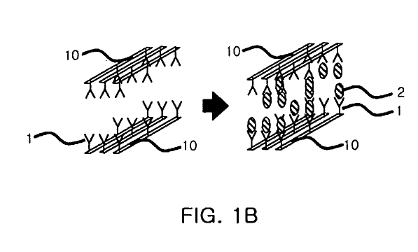

FIG. 1B is a schematic view illustrating 3-dimensional metallic nanowire gap electrodes used in a nanobiosensor according to one exemplary embodiment of the present invention.

Referring to FIG. 1B, in the bio-sensor including 3-dimensional metallic nanowire gap electrodes according to one exemplary embodiment of the present invention, a gap is formed between metallic nanowires 10 of upper and lower substrate blocks by disposing the metallic nanowires 10 on the upper substrate block on the metallic nanowires 10 on upper and lower substrate block so that they can be spaced apart from each other. According to the present invention, the probability that antigen binds to antibody may be enhanced at the presence of the gap (hereinafter, referred to as ‘3-dimensional metallic nanowire gap’) between the metallic nanowires 10 formed on the upper and lower substrate blocks, as well as the gap between the flat metallic nanowires. Also, the increase in the number of electrodes that may be electrically detected by the 3-dimensional metallic nanowire gap results in the enhanced sensor sensitivity.

More particularly, when sensing is executed using the bio-sensor including 3-dimensional metallic nanowire gap electrodes according to one exemplary embodiment of the present invention, a method for measuring the resistance, capacitance, inductance or impedance between the metallic nanowires 10 formed on the upper and lower substrate blocks, as well as method for measuring the resistance, capacitance, inductance or impedance between the metallic nanowires 10 formed on each substrate block may be used together with the bio-sensor, which leads to the enhanced sensor sensitivity.

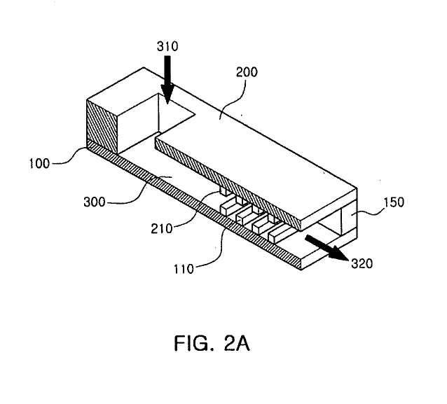

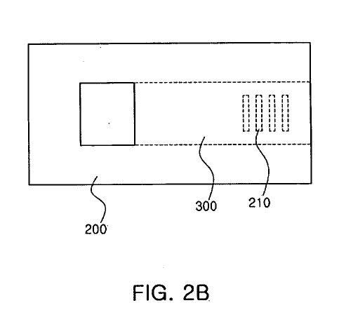

FIGS. 2A and 2B are a cross-sectional view and a top plane view, respectively, illustrating a bio-sensor including 3-dimensional metallic nanowire gap electrodes according to one exemplary embodiment of the present invention.

Referring to FIG. 2A, the bio-sensor including 3-dimensional metallic nanowire gap electrodes according to one exemplary embodiment of the present invention includes an upper substrate block 200 having a plurality of metallic nanowires 210 formed on a lower surface thereof and including an injection port 310 through which a biomaterial-containing sample is injected, a lower substrate block 100 having a plurality of metallic nanowires 110 formed on an upper surface thereof, and a supporting unit 150 supporting the upper and lower substrate blocks 200 and 100 so that the upper and lower substrate blocks 200 and 100 can be disposed spaced apart at a predetermined distance to form a nanochannel 300.

In this case, the metallic nanowires 210 and 110 are made of metals, such as Ag, Cu, Au, Al, Pt, and alloys thereof, having low electric resistance. Also, the supporting unit 150 may be formed between the upper substrate block 200 and the lower substrate block 100 and made of materials (for example, polymers) that are different from the flat upper and lower substrate blocks 200 and 100, or the supporting unit 150 may be formed integrally with the upper substrate block 200 or the lower substrate block 100.

As shown in FIG. 2B, the metallic nanowires 210 and 110 formed on the upper and lower substrate blocks are arranged overlapped with each other, and also arranged vertically to the nanochannel 300. That is, when the metallic nanowires 110 formed on the lower substrate block are viewed from the top, the metallic nanowires 110 are arranged so that they can be covered with the metallic nanowires 210 formed on the upper substrate block.

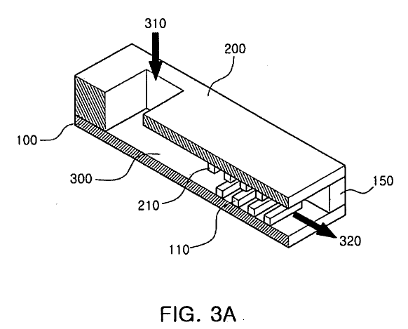

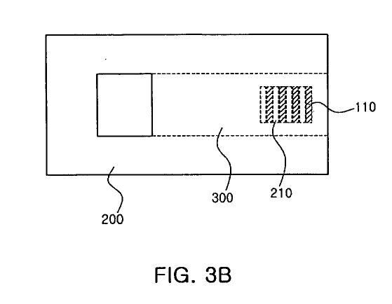

FIGS. 3A and 3B are a cross-sectional view and a top plane view, respectively, illustrating a bio-sensor including 3-dimensional metallic nanowire gap electrodes according to another exemplary embodiment of the present invention.

Referring to FIG. 3A, like the bio sensor according to one exemplary embodiment of the present invention, the bio sensor including 3-dimensional metallic nanowire gap electrodes according to another exemplary embodiment of the present invention also includes an upper substrate block 200 having a plurality of metallic nanowires 210 formed on a lower surface thereof and including an injection port 310 through which a biomaterial-containing sample is injected, a lower substrate block 100 having a plurality of metallic nanowires 110 formed on an upper surface thereof, and a supporting unit 150 supporting the upper and lower substrate blocks 200 and 100 so that the upper and lower substrate blocks 200 and 100 can be disposed spaced apart at a predetermined distance to form a nanochannel 300.

As shown in FIG. 3B, the metallic nanowires 210 and 110 formed on the upper and lower substrate blocks 200 and 100 are arranged vertically to the nanochannel 300, and also arranged alternately one by one. That is, when the metallic nanowires 110 formed on the lower substrate block are viewed from the top, the metallic nanowires 110 are arranged so that they can be viewed through gaps between a plurality of the metallic nanowires 210 formed on the upper substrate block.

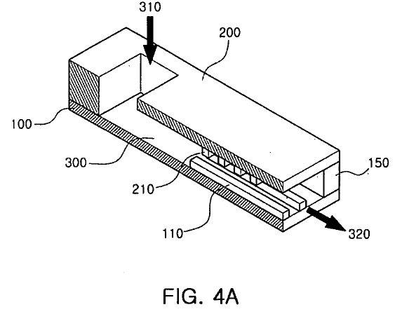

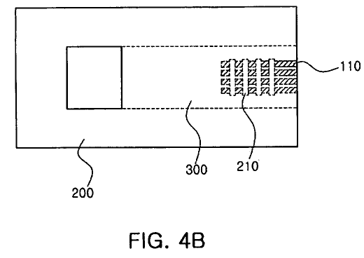

FIGS. 4A and 4B are a cross-sectional view and a top plane view, respectively, illustrating a bio-sensor including 3-dimensional metallic nanowire gap electrodes according to still another exemplary embodiment of the present invention.

Referring to FIG. 4A, like the bio sensor according to the exemplary embodiments of the present invention, the bio sensor including 3-dimensional metallic nanowire gap electrodes according to still another exemplary embodiment of the present invention also includes an upper substrate block 200 having a plurality of metallic nanowires 210 formed on a lower surface thereof and including an injection port 310 through which a biomaterial-containing sample is injected, a lower substrate block 100 having a plurality of metallic nanowires 110 formed on an upper surface thereof, and a supporting unit 150 supporting the upper and lower substrate blocks 200 and 100 so that the upper and lower substrate blocks 200 and 100 can be disposed spaced apart at a predetermined distance to form a nanochannel 300.

As shown in FIG. 4B, ones of the metallic nanowires 210 and 110 formed on the upper and lower substrate blocks 200 and 100, for example, the metallic nanowires 210 of the upper substrate block in the case of the still another exemplary embodiment of the present invention are arranged vertically to the nanochannel 300, and the other ones, for example, the metallic nanowires 110 of the lower substrate block are arranged horizontally with the nanochannel 300. That is, when the metallic nanowires 210 and 110 are viewed from the top, the metallic nanowires 210 of the upper substrate block and the metallic nanowires 110 of the lower substrate block are arranged so that they can be crossed vertically to each other.

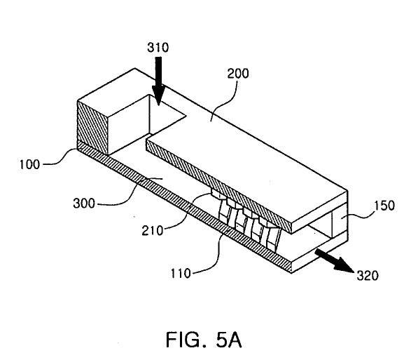

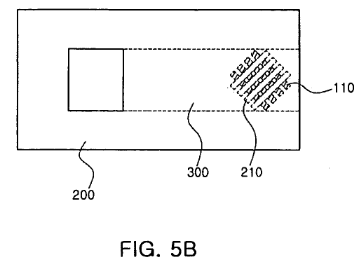

FIGS. 5A and 5B are a cross-sectional view and a top plane view, respectively, illustrating a bio-sensor including 3-dimensional metallic nanowire gap electrodes according to still another exemplary embodiment of the present invention.

Referring to FIG. 5A, like the bio sensor according to the exemplary embodiments of the present invention, the bio sensor including 3-dimensional metallic nanowire gap electrodes according to still another exemplary embodiment of the present invention also includes an upper substrate block 200 having a plurality of metallic nanowires 210 formed on a lower surface thereof and including an injection port 310 through which a biomaterial-containing sample is injected, a lower substrate block 100 having a plurality of metallic nanowires 110 formed on an upper surface thereof, and a supporting unit 150 supporting the upper and lower substrate blocks 200 and 100 so that the upper and lower substrate blocks 200 and 100 can be disposed spaced apart at a predetermined distance to form a nanochannel 300.

As shown in FIG. 5B, the metallic nanowires 210 and 110 formed on the upper and lower substrate blocks are neither arranged vertically to nor horizontally with the nanochannel 300, but arranged at a predetermined angle in respect to the nanochannel 300. That is, when the metallic nanowires 210 and 110 are viewed from the top, the metallic nanowires 210 of the upper substrate block and the metallic nanowires 110 of the lower substrate block are arranged so that they can form a predetermined angle (θ) in respect to each other.

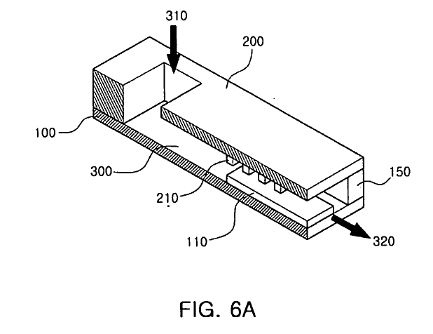

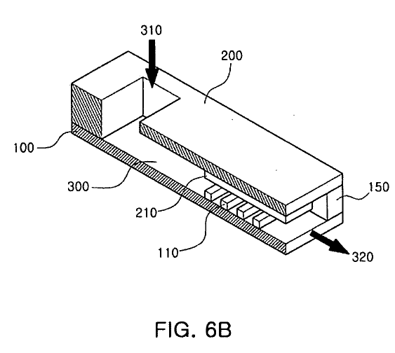

FIGS. 6A and 6B are a cross-sectional view and a top plane view, respectively, illustrating a bio-sensor including 3-dimensional metallic nanowire gap electrodes according to yet another exemplary embodiment of the present invention.

Referring to FIG. 6A, the bio sensor including 3-dimensional metallic nanowire gap electrodes according to yet another exemplary embodiment of the present invention includes an upper substrate block 200 having a plurality of metallic nanowires 210 formed on a lower surface thereof and including an injection port 310 through which a biomaterial-containing sample is injected, a lower substrate block 100 having metal electrode 110 formed on an upper surface thereof, and a supporting unit 150 supporting the upper and lower substrate blocks 200 and 100 so that the upper and lower substrate blocks 200 and 100 can be disposed spaced apart at a predetermined distance to form a nanochannel 300.

Referring to FIG. 6B, the bio sensor including 3-dimensional metallic nanowire gap electrodes according to yet another exemplary embodiment of the present invention also includes an upper substrate block 200 having metal electrode 210 formed on a lower surface thereof and including an injection port 310 through which a biomaterial-containing sample is injected, a lower substrate block 100 having a plurality of metallic nanowires 110 formed on an upper surface thereof, and a supporting unit 150 supporting the upper and lower substrate blocks 200 and 100 so that the upper and lower substrate blocks 200 and 100 can be disposed spaced apart at a predetermined distance to form a nanochannel 300.

That is, according to yet another exemplary embodiment of the present invention, only the electrodes formed on one of the upper and lower substrate blocks 200 and 100 are made of metallic nanowires, and the electrode formed on the other of the upper and lower substrate blocks 200 and 100 is composed of flat metal electrode.

According to the above-mentioned various exemplary embodiments of the present invention, the bio sensor including 3-dimensional metallic nanowire gap electrodes is composed of the upper and lower substrate blocks 200 and 100 including a plurality of metallic nanowires, and the supporting unit 150 supporting the upper and lower substrate blocks so that the upper and lower substrate blocks can be disposed spaced apart at a predetermined distance from each other. The biomaterial-containing sample to be detected enters through the injection port 310, and is passed through the 3-dimensional metallic nanowire gap electrode formed in nanochannel 300, and then discharged through the exhaust port 320. In this case, the sample introduced through the injection port 310 may be passed through the nanochannel 300 due to capillary phenomenon in the nanochannel 300 without performing an additional pumping operation.

Meanwhile, the metallic nanowires on the upper and lower substrate blocks 200 and 100 may be disposed in various manners, for example, disposed so that the upper and lower substrate blocks 200 and 100 can be arranged vertically to or horizontally with the nanochannel 300, or arranged at a predetermined angle in respect to the nanochannel 300, which makes it possible to relieve or prevent a blocking phenomenon that may appear in the front end of the nanochannel 300 according to the kind of samples to be detected.

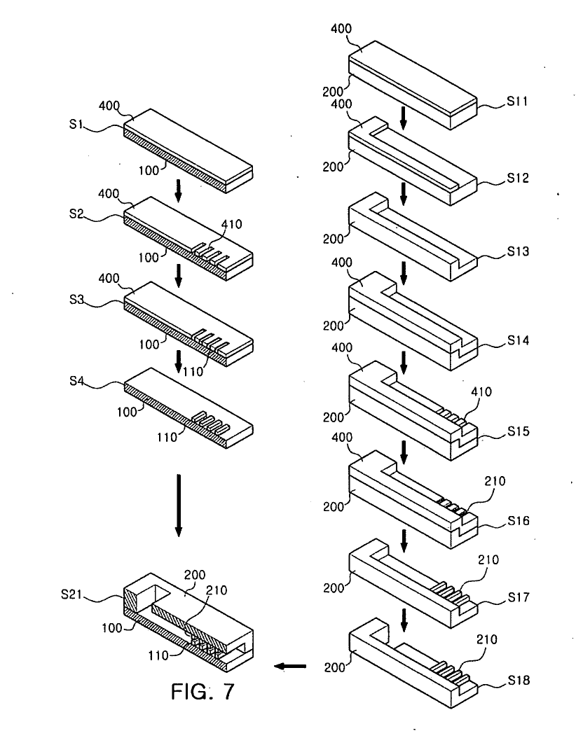

FIG. 7 is a flow chart illustrating a method of manufacturing a bio-sensor including 3-dimensional metallic nanowire gap electrodes according to one exemplary embodiment of the present invention.

First, a resist 400 is applied onto an upper surface of the lower substrate 100 (S1), and a metallic nanowire pattern 410 is formed on the resist 400, for example, using a nanopatterning method such as e-beam lithography or nanoimprinting techonologies (S2). In this case, the arrangement of the metallic nanowires 210 and 110 of the upper and lower substrates 200 and 100 and the nanochannel 300 may be widely varied, depending on the positions and arrangement of the metallic nanowire pattern 410. Then, a metal is deposited on the metallic nanowire pattern 410 of the lower substrate 100 to form metallic nanowires 110 on the lower substrate 100 (S3). In this case, the metallic nanowire 110 of the lower substrate 100 is made of a metal, such as Ag, Cu, Au, Al, Pt, and alloys thereof, having low electric resistance. Then, the resist 400 is removed from the lower substrate 100 (S4) to form a plurality of metallic nanowires 110 on an upper surface of the lower substrate 100.

Meanwhile, in the case of the upper substrate 200, a resist 400 is applied onto a lower surface of the upper substrate 200 (S11), and a nanochannel is then patterned on the resist 400 in order to determine a width and a length of the nanochannel 300 (S12). Then, the upper substrate 200 is etched using the patterned resist 400 as a mask (S13). In the etching process, it is possible to adjust the depth of the nanochannel 300. The etching may be carried out using one of processes such as chemical wet etching, vapor-phase etching (VPE), plasma etching and reactive ion etching (RIE). In the same manner as in the lower substrate 100, a plurality of metallic nanowires 210 are then formed on the lower surface of the upper substrate 200 by applying a resist 400 onto the lower surface of the upper substrate 200 (S14), forming a metallic nanowire pattern 410 on the resist 400 (S15), depositing a metal on the metallic nanowire pattern 410 of the upper substrate 200 (S16) and removing the resist 400 from the upper substrate 200 (S17). Then, an injection port 310 is formed to inject a sample to the upper substrate 200 (S18).

Finally, the upper and lower substrates 200 and 100 are arranged through optical methods or mechanical methods using the metallic nanowires 210 and 110 of the upper and lower substrates 200 and 100, and the arranged upper and lower substrates 200 and 100 are attached to each other (S21). The attaching of the upper and lower substrates 200 and 100 may be carried out using one of bonding processes such as anodic bonding, fusion bonding, bonding using polymer, and bonding using self-assembled monolayer (SAM).

A distance of the 3-dimensional metallic nanowire gap according to the present invention manufactured in the above-mentioned processes may be varied widely by adjusting the depth of the nanochannel 300 and the thickness of the metallic nanowires 210 and 110 deposited on the upper and lower substrates 200 and 100.

According to this exemplary embodiment, it is described that the metallic nanowires are deposited on the upper and lower substrates 200 and 100, but it may be considered that flat metal electrode is deposited on one of the upper and lower substrates 200 and 100 and a plurality of metallic nanowires are deposited on the other of the upper and lower substrates 200 and 100.

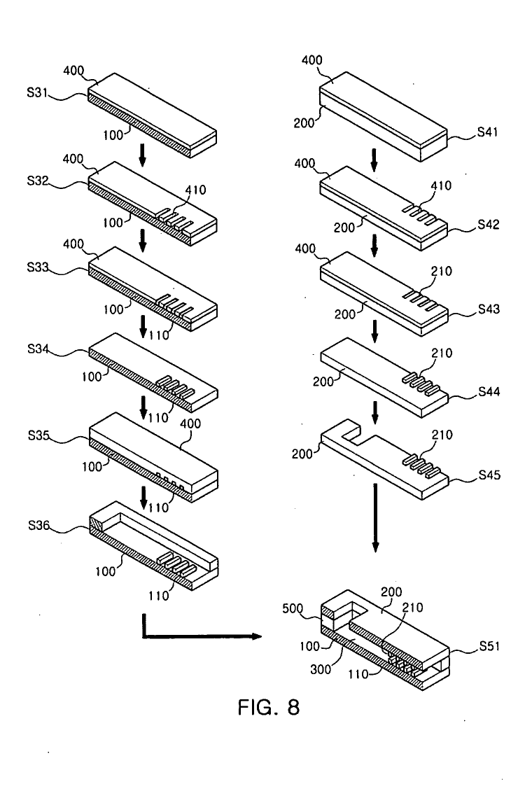

FIG. 8 is a flow chart illustrating a method of manufacturing a bio-sensor including 3-dimensional metallic nanowire gap electrodes according to another exemplary embodiment of the present invention.

First, a plurality of metallic nanowires 110 are formed on an upper surface of the lower substrate 100 using the above-mentioned method as shown in FIG. 7 (S31 to S34). Also, a plurality of metallic nanowires 210 are formed in a lower surface of the upper substrate 200 (S41 to S44), and an injection port 310 is then formed to inject a sample to the upper substrate 200 (S45).

Next, a polymer 500 is spin-coated onto the upper surface of the lower substrate 100, on which the metallic nanowires 110 are formed, to form a nanochannel 300 (S35). In this case, the thickness of the nanochannel 300 is determined according to the viscosity of the polymer 500, and the spin-coating RPM (revolutions per minute) and time.

Then, a width and a length of the nanochannel 300 are determined, and the polymer 500 is etched using a mask pattern (S36). The etching of the polymer 500 may be carried out using one of processes such as chemical wet etching, vapor-phase etching (VPE), plasma etching and reactive ion etching (RIE) processes.

Finally, the metallic nanowires 210 and 110 of the upper and lower substrates 200 and 100 are used to arrange the upper and lower substrates 200 and 100 by using an optical method or a mechanical method, and the arranged upper and lower substrates 200 and 100 are attached to each other (S51). The attaching of the upper and lower substrates 200 and 100 may be carried out using one of bonding processes such as anodic bonding, fusion bonding, bonding using polymer, and bonding using self-assembled monolayer (SAM).

A distance of the 3-dimensional metallic nanowire gap according to the present invention manufactured in the above-mentioned processes may be widely varied by adjusting the depth of the nanochannel 300 and the thickness of the metallic nanowires 210 and 110 deposited on the upper and lower substrates 200 and 100.

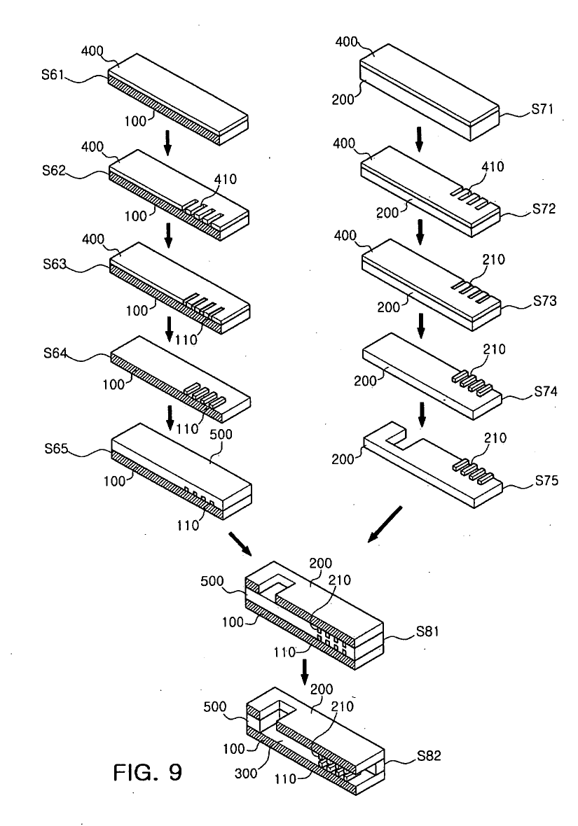

FIG. 9 is a flow chart illustrating a method of manufacturing a bio-sensor including 3-dimensional metallic nanowire gap electrodes according to still another exemplary embodiment of the present invention.

First, a plurality of metallic nanowires 110 are formed on an upper surface of the lower substrate 100 using the above-mentioned method as shown in FIG. 7 (S61 to S64). Also, a plurality of metallic nanowires 210 are formed in a lower surface of the upper substrate 200 (S71 to S74), and an injection port 310 is then formed to inject a sample to the upper substrate 200 (S75).

Then, a polymer 500 is spin-coated onto the upper surface of the lower substrate 100, on which the metallic nanowires 110 are formed, to form a nanochannel 300 (S65). In this case, the thickness of the nanochannel 300 is determined according to the viscosity of the polymer 500, and the spin-coating RPM and time.

Subsequently, the metallic nanowires 210 and 110 of the upper and lower substrates 200 and 100 are used to arrange the upper and lower substrates 200 and 100 by using an optical method or a mechanical method, and the arranged upper and lower substrates 200 and 100 are attached to each other (S81). The attaching of the upper and lower substrates 200 and 100 may be carried out using one of bonding processes such as anodic bonding, fusion bonding, bonding using polymer, and bonding using a self-assembled monolayer (SAM).

Finally, a width and a length of the nanochannel 300 are determined, and the polymer 500 on the upper and lower substrates 200 and 100, which are attached to each other using the polymer 500 as the supporting unit as described above, is exposed to UV radiation using a mask pattern, and the exposed polymer 500 is removed through the injection port 310 and the exhaust port 320 (S82).

A distance of the 3-dimensional metallic nanowire gap according to the present invention manufactured in the above-mentioned processes may be widely varied by adjusting the depth of the nanochannel 300 and the thickness of the metallic nanowires 210 and 110 deposited on the upper and lower substrates 200 and 100.

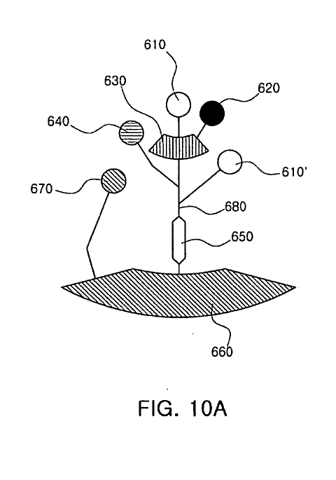

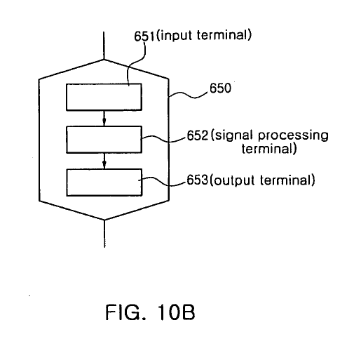

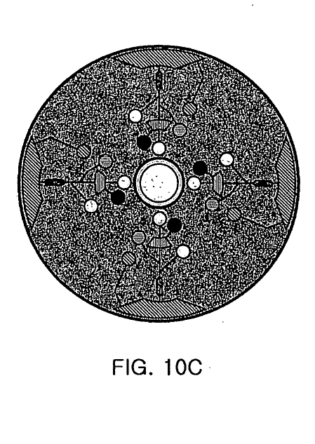

FIG. 10A is a configurational view illustrating a bio-disk system including the bio-sensor manufactured according to one exemplary embodiment of the present invention.

Referring to FIG. 10A, the bio-disk system according to one exemplary embodiment of the present invention includes a bio-sensor 650 including 3-dimensional metallic nanowire gap electrodes, buffer injection chambers 610 and 610′, a sample injection chamber 620, a pretreatment chamber 630, a calibrant injection chamber 640, an exhaust chamber 660, an exhaust port 670 and a microchannel 680. Here, these components are disposed in a disk-type body to form a lab-on-a-chip (LOC).

An operation principle of the bio-disk system will be described in more detail, as follows.

First, when a biomaterial-containing sample to be detected is injected into the sample injection chamber 620 of the bio-disk system and the bio-disk system is rotated, the bio-sensor 650 is washed with a buffer that flows out from the lower buffer injection chamber 610′ due to centrifugal force of the bio-disk system, and the signal origin of the bio-sensor 650 is then compensated for by a calibrant solution that flows out from the calibrant injection chamber 640.

At the same time, the pretreatment chamber 630 is washed with a buffer that flows out from the upper buffer injection chamber 610, and the sample injected into the sample injection chamber 620 flows out to the pretreatment chamber 630, followed by undergoing a pretreatment process. In this case, the sample is subject to a suitable pretreatment process, depending on the kind of the biomaterials to be detected in the bio-disk system.

Then, the sample pretreated in the pretreatment chamber 630 flows out to the bio-sensor 650 through the microchannel 680, and is then detected by the bio-sensor including 3-dimensional metallic nanowire gap electrodes according to one exemplary embodiment of the present invention.

Subsequently, the detected sample is all collected in the exhaust chamber 660 and discharged through the exhaust port 670.

FIG. 10B is a configurational view illustrating a bio-sensor including the metallic nanowire gap electrodes manufactured according to one exemplary embodiment of the present invention.

Referring to FIG. 10B, the bio-sensor 650 included in the bio-disk system is mainly composed of an input terminal 651, a signal processing terminal 652 and an output terminal 653. The input terminal 651 includes the bio-sensor according to one exemplary embodiment of the present invention, the signal processing terminal 652 processes signals from electrodes of the input terminal 651, and the output terminal 653 outputs the signals processed in the signal processing terminal 652 in the form of electric, magnetic or optical signals.

FIG. 10C is a plane view illustrating that the bio-disk system as shown in FIG. 10A is arranged on a disk-type body.

The one or more above-mentioned bio-disk system may be arranged on the disk-type body, as shown in FIG. 10C. In this case, the disk-type body may include thin disk-type bodies such as CD-ROMs, DVDs, bio CDs and bio DVDs.

As described above, the bio-sensor including 3-dimensional metallic nanowire gap electrodes, which is manufactured by forming metallic nanowires on the upper and lower substrates, arranging the upper and lower substrates using the metallic nanowires and attaching the upper and lower substrates to each other, may be useful to detect the biomaterial in high-sensitivity and high-efficiency manners by sensing electrical signals in real time, and to detect the biomaterial from a low-density sample or a small amount of a sample by the enhanced probability that a biomaterial is in contact with a sensor block detecting the biomaterial. Also, the bio-disk system manufactured using the bio-sensor may be useful to provide a self-diagnosis method in the home since the manufacturing cost of the self-diagnosis system is very inexpensive and the self-diagnosis system may be easily assembled at home using conventional optical disks, etc.

While the present invention has been shown and described in connection with the exemplary embodiments thereof and the accompanying drawings, it will be apparent to those skilled in the art that modifications and variations can be made without departing from the scope of the invention as defined by the appended claims.

Therefore, it should be understood that the scope of the invention is not defined by the detailed description and the drawings of the present invention but defined by the appended claims.

What is claimed is:

1. A bio-sensor, comprising:
an upper substrate block having a plurality of metallic nanowires formed on a lower surface thereof and including an injection port through which a biomaterial-containing sample is injected;
a lower substrate block having a plurality of metallic nanowires formed on an upper surface thereof; and
a supporting unit supporting the upper and lower substrate blocks so that the upper and lower substrate blocks are disposed spaced apart at a predetermined distance to form a nanochannel,
wherein the metallic nanowires formed on the upper and lower substrate blocks are combined to form 3-dimensional metallic nanowire gap electrodes.

2. The bio-sensor of claim 1, wherein the metallic nanowires formed on the upper and lower substrate blocks are arranged vertically to the nanochannel.

3. The bio-sensor of claim 2, wherein the metallic nanowires formed on the upper and lower substrate blocks are arranged overlapped with each other.

4. The bio-sensor of claim 2, wherein the metallic nanowires formed on the upper and lower substrate blocks are arranged alternately one by one.

5. The bio-sensor of claim 1, wherein one of the metallic nanowires formed on the upper and lower substrate blocks is arranged vertically to the nanochannel, and the other of the metallic nanowires are arranged horizontally with the nanochannel.

6. The bio-sensor of claim 1, wherein the metallic nanowires formed on the upper and lower substrate blocks are arranged at a predetermined angle in respect to each other.

7. The bio-sensor of claim 1, wherein the metallic nanowires formed on the upper and lower substrate blocks are made of at least one selected from the group consisting of Ag, Cu, Au, Al, Pt, and alloys thereof.

8. A bio-sensor, comprising:
an upper substrate block having a metal electrode formed on a lower surface thereof and including an injection port through which a biomaterial-containing sample is injected;
a lower substrate block having a metal electrode formed on an upper surface thereof; and
a supporting unit supporting the upper and lower substrate blocks so that the upper and lower substrate blocks are disposed spaced apart at a predetermined distance to form a nanochannel,
wherein one of the metal electrodes formed on the upper and lower substrate blocks is formed of a plurality of metallic nanowires, and
the metal electrodes formed on the upper and lower substrate blocks are combined to form 3-dimensional metallic nanowire gap electrodes.

9. The bio-sensor of claim 8, wherein the metal electrodes formed in the upper and lower substrate blocks are made of at least one selected from the group consisting of Ag, Cu, Au, Al, Pt, and alloys thereof.

10. A method of manufacturing a bio-sensor, the method comprising:
(a) forming a metal electrode on an upper surface of a lower substrate;
(b) patterning a nanochannel on a resist to determine a width and a length of the nanochannel, the resist being applied onto a lower surface of an upper substrate;
(c) etching the nanochannel using, as a mask, the pattern formed in operation (b);
(d) forming a metal electrode on the nanochannel formed in operation (c);
(e) arranging the upper and lower substrates using the metal electrodes formed on the upper and lower substrates; and
(f) attaching the upper and lower substrates arranged in operation (e).

11. The method of claim 10, wherein at least one of the metal electrodes formed on the upper and lower substrates comprises a plurality of metallic nanowires.

12. A method of manufacturing a bio-sensor, the method comprising:
(a) forming a plurality of metallic nanowires on an upper surface of a lower substrate;
(b) forming a plurality of metallic nanowires on a lower surface of an upper substrate;
(c) spin-coating a polymer onto the upper surface of the lower substrate to form a nanochannel;
(d) determining a width and a length of the nanochannel and etching the polymer using a mask pattern;
(e) arranging the upper and lower substrates using the metallic nanowires formed on the upper and lower substrates; and
(f) attaching the upper and lower substrates arranged in operation (e).

13. A method of manufacturing a bio-sensor, the method comprising:
(a) forming a plurality of metallic nanowires on an upper surface of a lower substrate;
(b) forming a plurality of metallic nanowires on a lower surface of an upper substrate;
(c) spin-coating a polymer on the upper surface of the lower substrate to form a nanochannel;
(d) arranging the upper and lower substrates using the metallic nanowires formed on the upper and lower substrates;
(e) attaching the upper and lower substrates arranged in operation (d); and
(f) determining a width and a length of the nanochannel and removing the polymer by UV exposure using the mask pattern.

14. The method of claim 12, wherein the etching of the nanochannel is performed using one process selected from the group consisting of chemical wet etching, vapor-phase etching (VPE), plasma etching and reactive ion etching (RIE) processes.

15. The method of claim 12, wherein the attaching of the upper and lower substrates is performed using one bonding process selected from the group consisting of anodic bonding, fusion bonding, bonding using polymer, and bonding using a self-assembled monolayer (SAM).

16. The method of claim 12, wherein the gaps between either the metallic nanowires or the metal electrodes formed in the upper and lower substrates are set to different distances by adjusting the depth of the nanochannel and the thickness of the deposited metallic nanowires or metal electrodes.

17. A bio-disk system for detecting a biomaterial from an injected sample using the bio-sensor as defined in claim 1.

18. The bio-disk system of claim 17, wherein the bio-sensor is disposed in a thin disk-type body selected from the group consisting of CD-ROMs, DVDs, bio CDs and bio DVDS.

19. The method of claim 13, wherein the etching of the nanochannel is performed using one process selected from the group consisting of chemical wet etching, vapor-phase etching (VPE), plasma etching and reactive ion etching (RIE) processes.

20. The method of claim 13, wherein the attaching of the upper and lower substrates is performed using one bonding process selected from the group consisting of anodic bonding, fusion bonding, bonding using polymer, and bonding using a self-assembled monolayer (SAM).

21. The method of claim 13, wherein the gaps between either the metallic nanowires or the metal electrodes formed in the upper and lower substrates are set to different distances by adjusting the depth of the nanochannel and the thickness of the deposited metallic nanowires or metal electrodes.
